(19) United States
(12) Patent Application Publication
(10) Pub. No.: US 2017/0119760 A1
(43) Pub. Date: May 4, 2017

(54) USE OF MASITINIB FOR THE TREATMENT OF PROGRESSIVE SUPRANUCLEAR PALSY

(71) Applicant: AB SCIENCE, Paris (FR)

(72) Inventors: Alain MOUSSY, Paris (FR); Jean-Pierre KINET, Lexington, MA (US); Colin MANSFIELD, Ecully (FR)

(21) Appl. No.: 15/337,992

(22) Filed: Oct. 28, 2016

(30) Foreign Application Priority Data
Oct. 28, 2015 (EP) 15 306 720.2

(51) Int. Cl. A61K 31/496; A61K 45/06; A61K 9/00

(52) U.S. Cl. CPC A61K 31/496; A61K 9/0053; A61K 45/06

(57) ABSTRACT

A mast cell inhibitor, a pharmaceutical composition and a method for treating patients afflicted with Progressive Supranuclear Palsy (PSP), wherein the patients are treated with a tyrosine kinase inhibitor, c-Kit inhibitor or mast cell inhibitor, in particular masitinib or a pharmaceutically acceptable salt or solvate thereof, optionally in combination with at least one pharmaceutically active ingredient.

### FIELD OF INVENTION

The present invention relates to a mast cell inhibitor, a pharmaceutical composition and a method for treating patients afflicted with Progressive Supranuclear Palsy (PSP), wherein said patients are treated with a tyrosine kinase inhibitor or mast cell inhibitor, in particular masitinib or a pharmaceutically acceptable salt or solvate thereof, optionally in combination with at least one pharmaceutically active ingredient.

### BACKGROUND OF INVENTION

Progressive supranuclear palsy, also known as Steele-Richardson-Olszewski syndrome, is a rare disease that involves the gradual deterioration of parts of the brain, i.e. neurodegeneration. PSP is typically described as a tauopathy; a class of neurodegenerative diseases associated with the pathological aggregation of tau protein in the human brain wherein tau protein is deposited within neurons in the form of neurofibrillary tangles (NFTs).

PSP causes serious and progressive problems with control of gait and balance, along with complex eye movement and thinking problems. Symptoms include loss of balance with unexplained falls, stiffness, difficulty moving the eyes (particularly up and down), difficulty swallowing, personality changes and dementia (loss of intellectual function). Most cases of PSP first develop in people who are 60-65 years of age, although the condition has been diagnosed in people as young as 40, with both genders being nearly equally affected.

Five clinical variants of PSP have been described with clinicopathological correlations: Classical PSP (Richardson's syndrome), and four atypical variants of PSP including PSP-Parkinsonism (PSP-P), PSP-Pure akinesia with gait freezing (PSP-PAGF), PSP-corticobasal syndrome (PSP-CBS), and PSP-progressive non fluent aphasia (PSP-PNFA).

Classical PSP (Richardson's syndrome) is the most common clinical variant and manifests with a lurching gait, falls due to postural instability, cognitive impairment and slowing of vertical saccadic eye movements. Progressively, patients develop other problems such as problems in speech and eventually a supranuclear gaze palsy and difficulties in swallowing. PSP-Parkinsonism (PSP-P) is characterized by prominent early parkinsonism (tremor, limb bradykinesia, axial and limb rigidity) rather than falls and cognitive change. Over the years, patients ultimately develop clinical features characteristic of Richardson's syndrome. PSP-Pure akinesia with gait freezing (PSP-PAGF) is characterized by progressive freezing of gait, speech and writing early in the course of the disease. Later, axial rigidity, and facial immobility can occur, and supranuclear downgaze paresis may emerge after a decade. PSP-corticobasal syndrome (PSP-CBS) is characterized by progressive, asymmetric dyspraxia, limb rigidity, bradykinesia and progressive postural instability. PSP-progressive non fluent aphasia (PSP-PNFA) is characterized by speech anomalies (apraxia of speech, agrammatism, phonemic errors). Motor symptoms appear later in the course of the disease.

Over time, the initial symptoms of PSP become more severe and debilitating in nature. The loss of balance can be so severe that walking becomes impossible and a wheelchair will eventually be required. As PSP progresses to an advanced stage the condition becomes life-threatening. Many people with PSP will need to consider using a feeding tube and typically also develop problems with their bowel and bladder functions. Because of dysphagia, people with PSP often experience repeated chest infections caused by fluids or small particles of food passing into their lungs, i.e. aspiration pneumonia, which is a leading cause of death in cases of PSP.

The National Institute of Neurological Disorders and Stroke (NINDS) and Society for Progressive Supranuclear Palsy criteria for PSP require a “gradually progressive” course for both possible and probable PSP. PSP patients typically have a life expectancy of 5 to 7 years, sometimes longer, and the rate of decline in PSP is estimated at approximately 12 PSPRS (Progressive Supranuclear Palsy Rating Scale) points per year [Golbe L I, et al. Brain. 2007 June; 130(Pt 6):1552-65]; [Boxer A L et al. Lancet Neurol. 2014 July; 13(7):676-85]. It has also been shown that the Richardson syndrome (PSP-RS) and PSP-parkinsonism (PSP-P) variants of PSP have different mean survival times of 6.8 years and 11.2 years, respectively [Jecmenica-Lukic M, et al. J Neurol. 2014 August; 261(8):1575-83]. The corresponding 5-year survival probabilities were 66% and 90%, respectively. Furthermore, there are also cases of PSP with more rapid progression (disease duration of 2 to 3 years) [Armstrong M J, et al. (2014) Movmnt Disords Clncl Practice, 1: 70-72]. Hence, there are at least two distinct subpopulations of PSP patients according to the rate of disease progression; these subpopulations being designated as either “normal progressors” or “faster progressors”, with the latter representing a more aggressive form of disease.

It is hypothesized that the different rates of disease progression reflect differing degrees of neuroinflammatory contribution to the pathogenesis of PSP, with ramifications as to the efficacy of therapies directed towards this mechanism of disease.

PSP is a debilitating and life-threatening disease that leads to a progressive inability to move and poor prospects of long-term survival. At the time being, there is no cure for PSP so the aim of treatment is to help controlling the symptoms. The use of anti-tauopathy medications and neurotransmitter replacement therapies, including a precursor of a catecholamine, such as dopamine, norepinephrine (noradrenaline), or epinephrine (adrenaline), such as for example levodopa; dopamine agonists, amantadine, tricyclic antidepressants, anticholinergics and selective serotonin reuptake inhibitors, has been shown to be largely ineffective and caused frequent adverse effects in patients with PSP. Electroconvulsive therapy for PSP has been shown to be of limited use with long hospitalization and significant treatment-induced confusion. Lack of efficacy and poor tolerance of these treatment options in PSP is unsatisfactory.

Therefore, there is still a need for effective drugs in the treatment of PSP.

### AIMS OF THE INVENTION

The invention aims to solve the technical problem of providing an active ingredient for the treatment of PSP.

The invention also aims to solve the technical problem of providing an active ingredient for an efficient treatment of PSP, especially in human patients.

The invention also aims to solve the technical problem of providing an active ingredient that improves prior art methods for the treatment of PSP.

The invention aims to provide an efficient treatment for PSP at an appropriate dose, route of administration, and daily intake.

### SUMMARY OF THE INVENTION

The Progressive Supranuclear Palsy Rating Scale (PSPRS) [Golbe L I, Ohman-Strickland P A A clinical rating scale for progressive supranuclear palsy. Brain. 2007 June; 130(Pt 6):1552-65. Epub 2007 Apr. 2] evaluates aspects of the disease in the domains of health history, mentation, bulbar function, eye and lid movement, limb movement, and trunk movement. The total maximum score is 100, reflecting the highest level of impairment. The Progressive Supranuclear Palsy Staging System [Golbe L I and the Medical Advisory Board of the Society for Progressive Supranuclear Palsy. A clinical rating scale and staging system for progressive supranuclear palsy. Neurology 1997; 48 (Suppl): A326] describes five disease stages. Details of the PSPRS and PSP Staging System are available from various published sources including the appendix of Zampieri et al. 2006 [Zampieri C, et al. Phys Ther. 2006 June; 86(6):870-80].

In a particular embodiment, patients suffering from PSP are patients with PSP stage≦II defined by a Progressive Supranuclear Palsy Rating Scale (PSPRS) score inferior to 55. In a more particular embodiment, patients suffering from PSP are patients with PSP stage≦II defined by at least a 12-month history of postural instability or falls during the first 3 years that symptoms are present; a PSPRS score inferior to 55; and an akinetic-rigid syndrome with prominent axial rigidity.

In one embodiment, other criteria are measured to assess PSP treatment. In one embodiment, this other criterion is selected from the group consisting of: the Hachinski Ischaemic Score (HIS), Dementia of Alzheimer's Type (DAT), such as PSP, and Vascular Dementia (VaD). The HIS represents a clinical tool helpful in the differentiation of the commonest dementia types.

A HIS inferior or equal to 4, preferably inferior or equal to 3, may be related to PSP. A HIS superior or equal to 7 may be related to a vascular dementia. The score is obtained by adding the values according to Table 1 below.

TABLE 1
The Hachinski Ischaemic Score.

| Item No. | Description | Value |
|---|---|---|
| 1 | Abrupt onset | 2 |
| 2 | Stepwise deterioration | 1 |
| 3 | Fluctuating course | 2 |
| 4 | Nocturnal confusion | 1 |
| 5 | Relative preservation of personality | 1 |
| 6 | Depression | 1 |
| 7 | Somatic complaints | 1 |
| 8 | Emotional incontinence | 1 |
| 9 | History of hypertension | 1 |
| 10 | History of stroke | 2 |
| 11 | Associated atherosclerosis | 1 |
| 12 | Focal neurological symptoms | 2 |
| 13 | Focal neurological signs | 2 |

In one embodiment, the modified Hachinski will not include the focal neurological signs, symptoms or pseudobulbar affect questions (questions 8, 11 and 12), given the prominence of all 3 in PSP.

In one embodiment, the patients suffering from PSP have a mini-mental state examination (MMSE) Score superior or equal to 25 [Folstein M F, et al. (1975). Journal of psychiatric research 12 (3): 189-98]; [National Institute for Clinical Excellence (NICE) Technology Appraisal Guidance 217, 2011].

PSP patients typically have a life expectancy of 5 to 7 years, sometimes longer, and the rate of decline in PSP is estimated at approximately 12 PSPRS points per year [Golbe L I, et al. Brain. 2007 June; 130(Pt 6):1552-65]; [Boxer A L et al. Lancet Neurol. 2014 July; 13(7):676-85]. It has also been shown that the Richardson syndrome (PSP-RS) and PSP-parkinsonism (PSP-P) variants of PSP have different mean survival times of 6.8 years and 11.2 years, respectively [Jecmenica-Lukic M, et al. J Neurol. 2014 August; 261(8):1575-83]. The corresponding 5-year survival probabilities were 66% and 90%, respectively. Furthermore, there are also cases of PSP with more rapid progression (disease duration of 2 to 3 years) [Armstrong M J, et al. (2014) Movmnt Disords Clncl Practice, 1: 70-72]. Hence, there are at least two distinct subpopulations of PSP patients according to the rate of disease progression; these subpopulations being designated as either “normal progressors” or “faster progressors”. It is hypothesized that the different rates of disease progression reflect differing degrees of neuroinflammatory contribution to the pathogenesis of PSP, with implications as to the efficacy of therapies directed towards this mechanism of disease.

PSP patients whose progression before randomization is less than 1.3 points per month (as measured by progression of PSPRS) are considered ‘normal progressors’. A second population of PSP patients whose progression as measured by PSPRS before randomization is greater than or equal to 1.3 points per month are considered ‘faster progressors’, representing a more aggressive form of disease. The calculation of progression of PSPRS (point/month) is as follows:

{(score PSPRS at baseline)−(score PSPRS at date of first symptom)} divided by {(time between first symptom and randomization)}.

Since the score at date of first symptom can be assumed to be zero, this formula is simplified to:

{(score PSPRS at baseline)}divided by {(time between first symptom and randomization)}.

In one embodiment, patients suffering from PSP are patients diagnosed as suffering from the variant Richardson syndrome (PSP-RS).

In one embodiment, patients suffering from PSP are patients diagnosed as suffering from the variant PSP-parkinsonism (PSP-P).

In one embodiment, patients suffering from PSP are classified as ‘fast progressors’ or as having ‘aggressive PSP’, as defined by a progression of PSPRS of greater than or equal to 1.3 points per month.

In another embodiment, patients suffering from PSP are classified as ‘normal progressors’ or as having ‘non aggressive PSP’, as defined by a progression of PSPRS of less than 1.3 points per month.

The mechanism leading to cell death in PSP is unknown but is likely to be multifactorial, with both environmental and genetic influences playing a role. PSP is characterized neuropathologically by neuronal loss, gliosis with astrocytic plaques and accumulation of tau-immunoreactive NFTs in specific brain areas. It is thought that these NFTs cause the gradual deterioration of brain tissue seen in these patients. Differences in the rate and areas of accumulation of phosphorylated tau protein correlate with the five clinical variants of PSP. The factors that initiate tau-neurodegeneration are unknown.

Oxidative injury and inflammation are also thought to contribute to development and/or progression of PSP. Microglial activation is greater in PSP than in control brains, and microglial activation correlates with tau burden in most areas. Evidence for activated glia involvement in PSP has raised the possibility that neuroinflammation may contribute to its pathogenesis. Fernandez-Botrán and colleagues [Fernandez-Botran R. et al. Parkinsonism Relat Disord. 2011 November; 17(9):683-8] showed that there was a disease-specific topographical relationship among the expression of certain cytokines (IL-1β and TGFβ), microglial activation and neurodegenerative changes, suggesting that these cytokines may contribute to the pathologic process.

It has been discovered that a tyrosine kinase inhibitor, c-Kit inhibitor, or mast cell inhibitor such as masitinib is useful in the treatment of PSP as exemplified by results obtained on an animal model relevant to PSP in human.

Mast cells, which are found on both sides of the blood-brain barrier (BBB), play an important role in sustaining the inflammatory network [Theoharides T C. Mast cells and stress—a psychoneuroimmunologic al perspective. J Clin Psychopharmacol. 2002 April; 22(2):103-8] [Stassen M, et al. Arch Immunol Ther Exp (Warsz). 2002; 50(3):179-85] [Kinet J P. Immunol Rev. 2007 June; 217:5-7]. Moreover, it has been shown that mast cells are able to cross the BBB and their numbers may rapidly increase in response to physiological manipulations [Nautiyal K, et al. Proc Natl Acad Sci USA. 2008 Nov. 18; 105(46): 18053-18057] [Theoharides T C, et al. J Neuroimmunol. 2004 January; 146(1-2):1-12] [Silverman A J, et al. J Neurosci 2000, 20:401-408]. Hence, mast cells may actively participate in the pathogenesis of PSP, in part because they release large amounts of proinflammatory mediators that sustain the inflammatory network of the central nervous system.

Perivascular localized mast cells secrete numerous vasoactive molecules that regulate BBB permeability [Secor V H, et al. J Exp Med. 2000 Mar. 6; 191(5):813-22] [Esposito P, et al. J Pharmacol Exp Ther. 2002; 303:1061-1066] [Esposito P, et al. Brain Res. 2001 Jan. 5; 888(1):117-127] [Zhuang X, et al. J Neurobiol. 1996 December; 31(4):393-403] Inhibition of mast cell mediators and apoptosis of mast cells localized at the BBB would effectively reduce BBB permeability, thereby reinforcing its integrity and stemming the accumulation of exogenous damaging factors in the brain.

Increased permeability of the BBB (Brain-Blood-Barrier) is an established event associated with clinical and pathological signs of neurodegenerative disease. Indeed, BBB dysfunction is also a factor in PSP [Bartels A L, et al. (2008). J Neural Transm 115(7):1001-1009]; [Blair L J, et al. Acta Neuropathol Commun 2015 Jan. 31; 3:8].

It has been found according to the present invention that a tyrosine kinase inhibitor, c-Kit inhibitor, or mast cell inhibitor, in particular masitinib or a pharmaceutically acceptable salt or solvate thereof, is useful in the treatment of PSP.

In one embodiment, said tyrosine kinase inhibitor, c-Kit inhibitor, or mast cell inhibitor is administered in combination with at least one pharmaceutically active ingredient.

Said pharmaceutically active ingredient is preferably active in the treatment of neurodegenerative tauopathies. Said pharmaceutically active ingredient is preferably chosen from the group consisting of: levodopa, carbidopa-levodopa, dopamine agonists, monoamine oxidase B (MAO-B) inhibitors, catechol-O-methyltransferase (COMT) inhibitors, NMDA receptor antagonists, acetylcholinesterase inhibitors, and mixture thereof.

The present invention thus relates to a method for the treatment of PSP in a mammal, and especially a human patient, wherein said method comprises administering to a human patient in need thereof, a tyrosine kinase inhibitor, c-Kit inhibitor, or mast cell inhibitor, especially masitinib or a pharmaceutically acceptable salt or solvate thereof, optionally combined with at least one pharmaceutically active ingredient.

The invention also relates to a pharmaceutical composition or kit comprising a tyrosine kinase inhibitor, c-Kit inhibitor, or mast cell inhibitor, in particular masitinib or a pharmaceutically acceptable salt or solvate thereof, and at least one other pharmaceutically active ingredient, for use in a method for the treatment of PSP as defined according to the present invention.

The invention also relates to the use of a tyrosine kinase inhibitor, c-Kit inhibitor, or mast cell inhibitor, in particular masitinib or a pharmaceutically acceptable salt or solvate thereof, for the preparation of a medicament, or a pharmaceutical composition, for the treatment of PSP, optionally in combination with at least one other pharmaceutically active ingredient.

### DESCRIPTION OF THE INVENTION

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation. A tyrosine kinase inhibitor is a drug that inhibits tyrosine kinases, thereby interfering with signaling processes within cells. Blocking such processes can stop the cell growing and dividing or inhibit cell activity.

In one embodiment, the tyrosine kinase inhibitor of the invention has the following formula [A]:

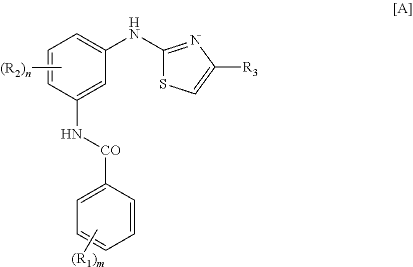

wherein R₁ and R₂, are selected independently from hydrogen, halogen, a linear or branched alkyl, cycloalkyl group containing from 1 to 10 carbon atoms, trifluoromethyl, alkoxy, cyano, dialkylamino, and a solubilizing group,
m is 0-5 and n is 0-4;
the group R₃ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;
(ii) a heteroaryl group such as 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;
or a pharmaceutically acceptable salt or solvate thereof.

Tyrosine kinase inhibitors of formula [A] can preferably be used as c-Kit inhibitors.

Unless otherwise specified, the below terms used herein are defined as follows:

As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C₆)aryl”.

As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3 -dimethylbutyl, 2,3-dimethylpentyl, 2,4-dimethylpentyl, 2,3 -dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.

As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

The term “heterocycle” as used herein, refers collectively to heterocycloalkyl groups and heteroaryl groups.

As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups include (but are not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein the term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —N—R₁₃, wherein r is 1 or 2; R₁₁ and R₁₂, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₁₁ and R₁₂ taken together with the nitrogen to which they are attached is optionally substituted heterocycloalkyl or optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

In certain embodiments, the term “substituent” or the adjective “substituted” refers to a solubilizing group.

The term “solubilizing group” means any group which can be substantially ionized and that enables the compound to be soluble in a desired solvent, such as, for example, water or water-containing solvent. Furthermore, the solubilizing group can be one that increases the compound or complex's lipophilicity. Typically, the solubilizing group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. For example, by “solubilizing group” it is referred herein to one of the following:
an alkyl, cycloalkyl, aryl, heretoaryl group comprising either at least one nitrogen or oxygen heteroatom or which group is substituted by at least one amino group or oxo group;
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl;
one of the structures a) to i) shown below, wherein the wavy line and the arrow line correspond to the point of attachment to core structure of Formula [A]:

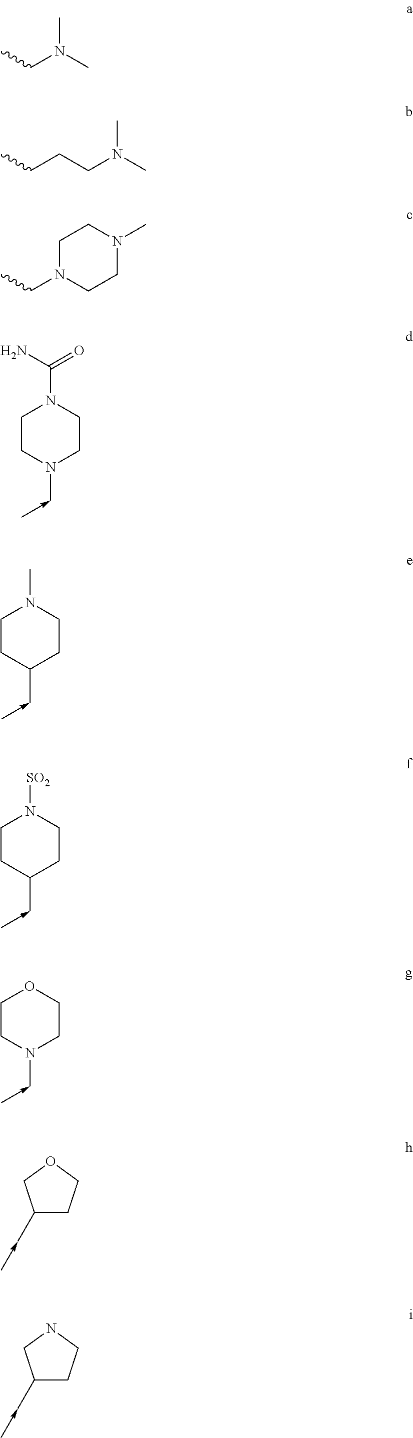

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

The term “halogen” means —F, —Cl, —Br or —I.

In a particular embodiment, the tyrosine kinase inhibitor of the invention has general formula [B],

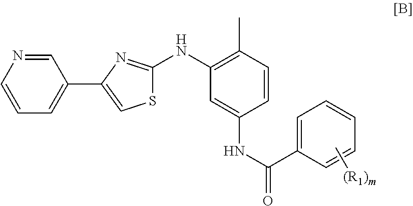

wherein:

R₁ is selected independently from hydrogen, halogen, a linear or branched alkyl, cycloalkyl group containing from 1 to 10 carbon atoms, trifluoromethyl, alkoxy, amino, alkylamino, dialkylamino, solubilizing group, and m is 0-5.

In one embodiment, the tyrosine kinase inhibitor, c-Kit inhibitor, or mast cell inhibitor is masitinib or a pharmaceutically acceptable salt or solvate thereof, more preferably masitinib mesilate.

Masitinib is a c-Kit/PDGFR/Lyn inhibitor with a potent anti-mast cell action. Masitinib is therefore a mast cell inhibitor.

New potent and selective tyrosine kinase inhibitors are 2-(3-aminoaryl)amino-4-aryl-thiazoles described in AB Science's PCT application WO 2004/014903.

Masitinib (AB1010) is a small molecule drug, selectively inhibiting specific tyrosine kinases such as c-Kit, PDGFR, Lyn, and Fyn without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) [Dubreuil et al., 2009, PLoS ONE 2009.4(9):e7258] [Davis et al., Nat Biotechnol 2011, 29(11): 1046-51]. The chemical name for masitinib is 4-(4-methylpiperazin-1-ylmethyl)-N-[4-methyl-3-(4-pyridin-3ylthiazol-2-ylamino) phenyl]benzamide—CAS number 790299-79-5, and the structure is shown below. Masitinib was first described in U.S. Pat. No. 7,423,055 and EP1525200B1. A detailed procedure for the synthesis of masitinib mesilate is given in WO 2008/098949.

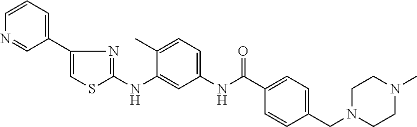 [SMILES: Cc1ccc(NC(=O)c2ccc(CN3CCN(C)CC3)cc2)cc1Nc1nc(-c2cccnc2)cs1]

Masitinib's main kinase target is c-Kit, for which it has been shown to exert a strong inhibitory effect on wild-type and juxtamembrane-mutated c-Kit receptors, resulting in cell cycle arrest and apoptosis of cell lines dependent on c-Kit signaling [Dubreuil et al., 2009, PLoS ONE, 4(9):e7258]. In vitro, masitinib demonstrated high activity and selectivity against c-Kit, inhibiting recombinant human wild-type c-Kit with a half inhibitory concentration (IC50) of 200±40 nM and blocking stem cell factor-induced proliferation and c-Kit tyrosine phosphorylation with an IC50 of 150±80 nM in Ba/F3 cells expressing human or mouse wild-type c-Kit. In addition to its anti-proliferative properties, masitinib can also regulate the activation of mast cells through its targeting of Lyn and Fyn, key components of the transduction pathway leading to IgE induced degranulation [Gilfillan et al., 2006, Nat Rev Immunol, 6:218-230] [Gilfillan et al., 2009, Immunological Reviews, 228:149-169]. This can be observed in the inhibition of FccRI-mediated degranulation of human cord blood mast cells [Dubreuil et al., 2009, PLoS ONE;4(9):e7258]. Masitinib is also an inhibitor of PDGFR α and β receptors. Recombinant assays show that masitinib inhibits the in vitro protein kinase activity of PDGFR-α and β with IC50 values of 540±60 nM and 800±120 nM. In Ba/F3 cells expressing PDGFR-α, masitinib inhibited PDGF-BB-stimulated proliferation and PDGFR-α tyrosine phosphorylation with an IC50 of 300±5 nM.

The present invention relates to a method for the treatment of PSP in a mammal, and especially a human patient, wherein said method comprises administering to a human patient in need thereof, a tyrosine kinase inhibitor, c-Kit inhibitor, or mast cell inhibitor, especially masitinib or a pharmaceutically acceptable salt or solvate thereof, optionally combined with at least one pharmaceutically active ingredient.

The present invention relates to a method for the treatment of PSP wherein said method comprises administering to a mammal in need thereof, at least one tyrosine kinase inhibitor, c-Kit inhibitor, or mast cell inhibitor.

In one embodiment, said tyrosine kinase inhibitor, c-Kit inhibitor, or mast cell inhibitor is administered to a human patient.

In one embodiment, said tyrosine kinase inhibitor, c-Kit inhibitor, or mast cell inhibitor is administered to a human patient diagnosed as suffering from the variant Richardson syndrome (PSP-RS).

In one embodiment, said tyrosine kinase inhibitor, c-Kit inhibitor, or mast cell inhibitor is administered to a human patient diagnosed as suffering from the variant PSP-parkinsonism (PSP-P).

In one embodiment, said tyrosine kinase inhibitor, c-Kit inhibitor, or mast cell inhibitor is administered to a human patient having a progression of PSPRS of greater than or equal to 1.3 points per month.

In one embodiment, said tyrosine kinase inhibitor, c-Kit inhibitor, or mast cell inhibitor is administered to a human patient having a progression of PSPRS of less than 1.3 points per month.

In one embodiment, said tyrosine kinase inhibitor is an inhibitor of kinase activity selected from the tyrosine kinase activity of: c-Kit, Lyn, Syk, Btk and Fyn.

In one embodiment, said tyrosine kinase inhibitor is an inhibitor of kinase activity selected from the tyrosine kinase activity of: c-Kit and Lyn.

In one embodiment, said tyrosine kinase inhibitor is a selective inhibitor of mast cell function.

In one embodiment, said mast cell inhibitor is masitinib or a pharmaceutically acceptable salt or solvate thereof, more preferably masitinib mesilate.

In another embodiment, said mast cell inhibitor is imatinib (STI571, Novartis), more preferably imatinib mesilate. Therefore, in a particular embodiment, the invention relates to a method for the treatment of PSP in a mammal, and especially a human patient, comprising the administration of an effective amount of the compound known in the art as imatinib (STI571, CGP57148B): 4-[(4-Methyl-1-piperazinyl)methyl]-N-(4-methyl-3-{[4-(3-pyridinyl)-2-pyrimidinyl]amino}phenyl)benzamide. The preparation of this compound is described in example 21 of EP 564 409 and the form, which is particularly useful is described in WO 99/03854.

In another embodiment, the mast cell inhibitor can be selected from: midostaurin (PKC412; Novartis), dasatinib (BMS354825; Bristol-Myers Squibb), sunitinib (SU11248; Pfizer), nilotinib (AMN107; Novartis), axitinib (AG013736; Pfizer), pazopanib (Glaxo SmithKline), toceranib (SU11654; Pfizer), BLU-285 (Blueprint Medicines), bosutinib (SKI-606; Pfizer), ibrutinib (PCI-32765; Pharmacyclics), LAS189386 (Almirall R&D Center), DP-2618 (Deciphera Pharmaceuticals), fostamatinib (R788; Rigel), and cromolyn sodium.

In another embodiment, the mast cell inhibitor is chosen from the group consisting of: masitinib, imatinib, cromolyn sodium, midostaurin, BLU-285, bosutinib, ibrutinib, LAS189386, DP-2618, fostamatinib, nilotinib, dasatinib, sunitinib, axitinib, pazopanib, and toceranib, or pharmaceutically acceptable salts or solvates thereof.

In one embodiment, said tyrosine kinase inhibitor, c-Kit inhibitor, or mast cell inhibitor is administered in combination with at least one pharmaceutically active ingredient. Said pharmaceutically active ingredient is preferably active in the treatment of PSP or related neurodegenerative tauopathies. Said pharmaceutically active ingredient is preferably chosen from the group consisting of: levodopa, carbidopa-levodopa, dopamine agonists, monoamine oxidase B (MAO-B) inhibitors, catechol-O-methyl transferase (COMT) inhibitors, NMDA receptor antagonists, acetylcholinesterase inhibitors, and mixture thereof. The dopamine agonist is preferably chosen from: bromocriptine, pergolide, pramipexole, ropinirole, piribedil, cabergoline, apomorphine and lisuride. The MAO-B inhibitor is preferably chosen from: safinamide, selegiline and rasagiline. The COMT inhibitor is preferably chosen from: entacapone and tolcapone. The NMDA receptor agonist is preferably chosen from: amantadine and memantine. The acetylcholinesterase inhibitor is preferably chosen from: rivastigmine, donepezil, and galantamine. Therefore, in embodiment, said pharmaceutically active ingredient is chosen from the group consisting of: levodopa, carbidopa-levodopa, bromocriptine, pergolide, pramipexole, ropinirole, piribedil, cabergoline, apomorphine lisuride, safinamide, selegiline, rasagiline, entacapone, tolcapone, amantadine, memantine, rivastigmine, donepezil, galantamine, and mixture thereof.

In one embodiment, said tyrosine kinase inhibitor, c-Kit inhibitor, or mast cell inhibitor, in particular masitinib or a pharmaceutically acceptable salt or solvate thereof, is administered at a daily dose of between 1.5 to 9.0 mg/kg/day; for example, 1.5, 3.0, 4.5, 6.0, 7.5, or 9.0 mg/kg, more preferably 3.0, 4.5 or 6 mg/kg/day (mg per kg bodyweight per day).

In one embodiment said tyrosine kinase inhibitor, c-Kit inhibitor, or mast cell inhibitor, in particular masitinib or a pharmaceutically acceptable salt or solvate thereof, is dose escalated by increments of 1.5 mg/kg/day to reach a maximum of 9.0 mg/kg/day, more preferably 6 mg/kg/day. Each dose escalation is subjected to toxicity controls with an absence of any toxicity events permitting dose escalation to occur.

In one embodiment dose escalation of said tyrosine kinase inhibitor, c-Kit inhibitor, or mast cell inhibitor, in particular masitinib or a pharmaceutically acceptable salt or solvate thereof, occurs at any time-point after at least 4 weeks after the initial dose has been administered and prior to 26 weeks after the initial dose has been administered; for example at week-4, week-8, week-12, week-16, week-20, or week-24. As an example, said tyrosine kinase inhibitor, c-Kit inhibitor, or mast cell inhibitor, in particular masitinib or a pharmaceutically acceptable salt or solvate thereof, is initially administered per os, preferably in two daily intakes, at a dose of 3 mg/kg/day during 4 weeks, then 4.5 mg/kg/day during 4 weeks, and then 6 mg/kg/day thereafter. In another example, masitinib or a pharmaceutically acceptable salt or solvate thereof is initially administered per os, preferably in two daily intakes, at a dose of 4.5 mg/kg/day during 12 weeks, and then 6 mg/kg/day thereafter.

Any dose indicated herein refers to the amount of active ingredient as such, not to its salt form.

Given that the masitinib dose in mg/kg/day used in the described dose regimens refers to the amount of active ingredient masitinib, compositional variations of a pharmaceutically acceptable salt of masitinib mesilate will not change the said dose regimens.

In one embodiment, said tyrosine kinase inhibitor, c-Kit inhibitor, or mast cell inhibitor, in particular masitinib or a pharmaceutically acceptable salt or solvate thereof, is administered orally.

In one embodiment, said tyrosine kinase inhibitor, c-Kit inhibitor, or mast cell inhibitor, in particular masitinib or a pharmaceutically acceptable salt or solvate thereof, is administered once or twice a day.

In one embodiment, said tyrosine kinase inhibitor, c-Kit inhibitor, or mast cell inhibitor, in particular masitinib or a pharmaceutically acceptable salt or solvate thereof, is administered in combination with said at least one pharmaceutically active ingredient in a combined preparation for simultaneous, separate, or sequential use.

The invention also relates to a tyrosine kinase inhibitor, c-Kit inhibitor, or mast cell inhibitor, in particular masitinib or a pharmaceutically acceptable salt or solvate thereof, as defined according to the present invention, for use in a treatment of PSP.

The invention also relates to a tyrosine kinase inhibitor, c-Kit inhibitor, or mast cell inhibitor, in particular masitinib or a pharmaceutically acceptable salt or solvate thereof, as defined according to the present invention, for use in a treatment of PSP, in combination with at least pharmaceutically active ingredient, preferably chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof.

The invention also relates to a pharmaceutical composition or kit comprising a tyrosine kinase inhibitor, c-Kit inhibitor, or mast cell inhibitor, in particular masitinib or a pharmaceutically acceptable salt or solvate thereof, for use in a method for the treatment of PSP as defined according to the present invention.

In one embodiment, the pharmaceutical composition for use in a method for the treatment of PSP according to the present invention comprises a mast cell inhibitor, preferably masitinib or a pharmaceutically acceptable salt or solvate thereof, in combination with one or more pharmaceutically acceptable excipients.

The invention also relates to a pharmaceutical composition or kit comprising a tyrosine kinase inhibitor, c-Kit inhibitor, or mast cell inhibitor, in particular masitinib or a pharmaceutically acceptable salt or solvate thereof, and at least one other pharmaceutically active ingredient, preferably chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof.

The invention also relates to the use a tyrosine kinase inhibitor, c-Kit inhibitor, or mast cell inhibitor, in particular masitinib or a pharmaceutically acceptable salt or solvate thereof, for the preparation of a medicament, or a pharmaceutical composition, for the treatment of PSP, optionally in combination with at least one other pharmaceutically active ingredient, preferably chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof.

The terms “as defined according to the invention” refer to any embodiments or aspects of the invention alone or in combination without limitation, including any preferred embodiments and variants, including any embodiments and features relating to said tyrosine kinase inhibitor, c-Kit inhibitor, or mast cell inhibitor, in particular masitinib or a pharmaceutically acceptable salt or solvate thereof, the method of treatment of PSP, pharmaceutical compositions and any combination with other pharmaceutically active ingredient(s), preferably chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof. “Masitinib” designates also a pharmaceutically acceptable salt or solvate thereof, especially masitinib mesilate, even when not explicitly stated.

The tyrosine kinase inhibitor, c-Kit inhibitor, or mast cell inhibitor and the optional at least one pharmaceutically active ingredient, are administered in a dosage regimen that comprises a therapeutically effective amount.

In relation to the present invention, the term “treatment” (and its various grammatical forms) refers to preventing, curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

Advantageously, the use or method comprises a long term administration of an effective amount of said tyrosine kinase inhibitor, c-Kit inhibitor, or mast cell inhibitor, especially masitinib or a pharmaceutically acceptable salt or solvate thereof, over more than 3 months, preferably more than 6 months.

In one embodiment, the use or method comprises administering said tyrosine kinase inhibitor, c-Kit inhibitor, or mast cell inhibitor, especially masitinib or a pharmaceutically acceptable salt or solvate thereof, as first, second or third-line treatment of PSP in a mammal, and especially a human patient.

As is known to the person skilled in the art, various forms of excipients can be used, adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverizable or ingestible form, for example via the intramuscular, intravenous, subcutaneous, intradermal, oral, topical, rectal, vaginal, ophthalmic, nasal, transdermal or parenteral route. A preferred route is oral administration. The present invention notably covers the use of a compound according to the present invention for the manufacture of pharmaceutical composition.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

According to a particular embodiment, the composition of the invention is an oral composition.

In one embodiment, compositions according to the invention may be in the form of tablets.

In one embodiment, compositions according to the invention may comprise from 50 to 500 mg of said tyrosine kinase inhibitor or mast cell inhibitor, especially masitinib or a pharmaceutically acceptable salt thereof. More particularly, the composition may comprise from 100 to 500 mg of said tyrosine kinase inhibitor or mast cell inhibitor, especially masitinib or a pharmaceutically acceptable salt thereof, for example, 100, 200, 300, 400, or 500 mg.

The present invention is further illustrated by means of the following examples. The data presented in these examples, and also in parts of the patent Description, are in part taken from preliminary analysis and as such represent a close approximation to the final, validated dataset.

### EXAMPLES

### Example 1

### Effect of Masitinib on Mouse Model of Neurodegenerative Tauopathy

Preclinical data from a mouse model of relevance to PSP-like diseases provided proof-of-concept for masitinib's neuroprotective effect in PSP and also supported initiation of a randomized controlled phase 2b/3 human trial. Masitinib's neuroprotective effect in PSP was investigated using the MPTP (1-methyl-4-phenyl-1,2,3,6-tetrahydropyridine) mouse model. This in vivo model is of relevance to PSP-like diseases (i.e. neurodegenerative tauopathies) and importantly it is capable of demonstrating the mechanism of action under investigation, i.e. inhibition of neuroinflammatory response in a neurodegenerative tauopathy. MPTP is a toxin that produces the same marked depletion of striatal dopamine and destruction of dopaminergic neurons in the substantia nigra as is observed in PSP [Oh M, et al. J Nucl Med. 2012 March; 53(3):399-406. Oh, 2012]; [Hardman C D, et al. Exp Neurol. 1997 March; 144(1):183-92].

The implication of mast cells in the development of PSP is not well characterized. The present work was undertaken to evaluate the role of these inflammatory cells in this pathology and more specifically the neuroprotective effect of masitinib, a tyrosine kinase inhibitor that specifically targets mast cells survival, proliferation and activation, in an animal model of relevance to PSP (i.e. the MPTP mouse model, a model of neurodegenerative tauopathies). The survival of dopaminergic neurons was studied by the characterization of the tyrosine hydroxylase expression.

Mice were treated with masitinib or solvent alone starting day 7 before intoxication. Each treated group included 10 animals for the MPTP intoxicated groups and 5 animals for the controls. The loss of tyrosine hydroxylase expression, indicative of dopaminergic neuron destruction, was analyzed by western blotting, immunohistochemistry and ELISA assay.

1.1. Materials and Methods

1.1.1. Experimental Items
40 outbred, C57BL/6 male mice, 23-26 g (January; France);
1-Methyl-4-Phenyl-1,2,3,6-TetrahydroPyridine (MPTP) (Cat #: M0896; Sigma Aldrich; France);
Masitinib (AB Science);
Tween 80 (Cat #: P8074; Sigma Aldrich; France);
1,2 propanediol (Cat #: 398039; Sigma Aldrich; France);
0.9% saline, sterile (B. Braun);
Polyclonal Antibody Anti-Tyrosine Hydroxylase (Cat #AB 152; Millipore; France);
Antibody biotinylated goat anti rabbit IgG (Cat #111-65-003, Jakson immunoresearch);
Streptavidin peroxidase complex (Cat #E2886, Sigma Aldrich, France);
Z-Lys-SBZL substrate solution (Cat #C3641, Sigma Aldrich, France);
DTNB (Cat #D8130, Sigma Aldrich, France);
Abcys Histogreen substrate kit (Cat #E109, AbCys);
Amplex Red Monoamine Oxidase Assay kit (Cat # Al2214, Invitrogen).

1.1.2. Preparation of Masitinib

400 mg of masitinib was stored at room temperature. The day before treatment starts, masitinib was dissolved to 6.25 mg/ml in 10% Tween 80; 10% Isopropanediol; 80% water solution, and thereafter aliquoted and stored at −20° C. This stock solution was defrosted before treatment of mice. The mice were treated orally by gavage with 0.1 ml corresponding to 25 mg/kg in a 24 g mouse.

1.1.3. Preparation of MPTP Solution

One ampoule of 100 mg MPTP was dissolved in 20 ml NaCl 0.9% to obtain 20 mg/kg of MPTP in 100 μl injected by mouse.

1.1.4. Inoculation of Mice

C57BL/6J mice are intoxicated with MPTP using an acute protocol, as described hereafter. On the day of MPTP exposure each mouse received 4 injections of MPTP solution (20 mg/kg) at 2-hour intervals for 1 day.

1.1.5. Treatment of Mice

Masitinib was administered by oral route in a volume of 0.1 mL in the morning and in the afternoon. Dosing was done 2 times per day at 6-hour interval starting day 7 before intoxication and stopping 7 days after MPTP administration. On day 14, mice were deeply anesthetized with 12.5 mg/kg Xylasine (Bayer, France), 192 mg/kg Ketamine (Mérial, France) diluted in physiological saline solution. Before brain extraction mice were perfused with 0.9% NaCl 2.6 mM EDTA solution.

During the period of experiment, the animals were treated either with masitinib twice a day at 30 mg/kg (single-agent, masitinib control group) or at 5 mg/kg (test group) or with solvent (control group) using per os administration. The treatment started 7 days before MPTP administration and continued 7 days after.

1.1.6. Tissue and Protein Samples

After mouse sacrifice and dissection, the left brain was submerged in 4% paraformaldehyde in PBS (phosphate buffered saline) solution for paraffin embedding and then cut into 5 μM-thick coronal section. And the right brain was immediately frozen in nitrogen liquid for protein analysis. The proteins were extracted with the homogenizer ultra turrax IKA-T10 basic system and quantified using the BCA test (bicinchoninic acid assay).

1.1.7. Tyrosine Hydroxylase Western Blotting 30 μg of total brain protein were used for western blot analysis using 1:1000 dilution anti-tyrosine hydroxylase rabbit polyclonal antibody (Cat #AB 152, Millipore) followed by 1:10000 horseradish peroxidase-conjugated anti-rabbit antibody (Jakson ImmunoResearch). Antibody to actin (Cat #A5316, Sigma Aldrich) were used as loading control followed by 1:10000 horseradish peroxidase-conjugated anti-mouse antibody (Jakson ImmunoResearch) Immunoreactive bands were detected using enhanced chemiluminescent reagents (GE Healthcare, Amersham, UK). Signal intensity was calculated using the Image J software.

1.1.8. Tyrosine Hydroxylase ELISA Assay

The microliter plate wells were coated overnight at 4° C. with 5 μg of total brain protein extract. All unbound sites were blocked with a blocking buffer 10% FCS in PBS 1 hour at room temperature. Then, the anti-tyrosine hydroxylase rabbit polyclonal antibody, diluted 1:1000 was added for 2 hours at room temperature. After rinsing with PBS, the wells were incubated with biotinylated goat anti-rabbit IgG (1:250) for 1 hour at room temperature (Cat #111-65-003, Jakson immunoresearch) and followed by incubation with a streptavidin peroxidase complex for 30 minutes (1:250) at room temperature (Cat #E2886, Sigma Aldrich). Peroxidase staining was revealed using the manufacturer's instructions of the TMB ELISA kit detection.

1.1.9. Tyrosine Hydroxylase Immunohistochemistry

The 5 μM-thick coronal sections were deparaffinized and hydrated, Endogenous peroxydase were inhibited with a dual endogenous enzyme block solution (Cat #S2003, DakoCytonation). Non specific protein binding was blocked with 1% BSA in PBS, pH=7,4. Sections were incubated overnight at 4° C. with anti-tyrosine hydroxylase rabbit polyclonal antibody, diluted 1:1000 (Cat #AB152, Millipore). After rinsing with PBS, the sections were incubated with biotinylated goat anti-rabbit IgG (1:500) for 30 minutes at room temperature (Cat #111-65-003, Jakson immunoresearch) followed by incubation with a streptavidin peroxidase complex for 30 minutes (1:500) at room temperature (Cat #E2886, Sigma Aldrich). Peroxidase staining was obtained using the AbCys Histogreen substrate kit (Cat #E109, AbCys) and counterstained with a Neutral Red solution. After dehydration the sections were cover-slipped with Eukitt®.

1.1.10. Tryptase Enzymatic Colorimetric Assay

5 μg of total brain protein extract were added in microliter plate wells and the quantity of tryptase enzyme measured by densitometry at 410 nm after addition of Z-Lys-SBZL substrat solution (Cat #C3641, Sigma Aldrich) and DTNB (Cat #D8130, Sigma Aldrich).

1.1.11. Monoamine Oxidase Fluorometric Assay

5 μg of total brain protein extract were added in microliter plate wells and the monoamine oxidase activity measured using manufacturer's instructions (Cat #A12214, Invitrogen) by fluorometric method using excitation at 545 nm and using emission detection at 590 nm.

1.1.12. Statistical Comparison

Statistical comparison of two selected groups was done with Mann Whitney test and comparison of multiple groups was done with ANOVA and Turkey's multiple comparison tests.

1.2. Results

No mortality was observed 24 hours after treatment by the MPTP. Brain samples were collected after mice anesthesia and sacrifice.

The tyrosine hydroxylase expression was detected from total brain protein extract both by western blotting and by ELISA assays (Table 2 and Table 3, respectively).

A decrease of the tyrosine hydroxylase expression, indicating dopaminergic neuron destruction, was detected after MPTP treatment relative to the control group. This result validates the protocol.

TABLE 2
Relative expression of tyrosine hydroxylase in
total brain protein extract using western blotting
assay after treatment by MPTP and masitinib.

| | Relative average expression of |
|---|---|
| Treatment | tyrosine hydroxylase |
| Control | 100 |
| Masitinib 2 × 30 mg/kg | 90 |
| MPTP | 77 |
| MPTP + Masitinib 2 × 5 mg/kg | 91 |

TABLE 3
Tyrosine Hydroxylase in total brain protein extract using
ELISA assay after treatment by MPTP and masitinib.

| | Mean expression of |
|---|---|
| Treatment | tyrosine hydroxylase |
| Control | 0.275 |
| Masitinib 2 × 30 mg/kg | 0.225 |
| MPTP | 0.175 |
| MPTP + Masitinib 2 × 5 mg/kg | 0.215 |

A significant decrease of tyrosine hydroxylase expression was detected after MPTP treatment with respect to the control group. The treatment with masitinib alone did not modify significantly the basal expression of tyrosine hydroxylase compared with control. A significantly diminished decrease of tyrosine hydroxylase expression in response to MPTP treatment was observed in the masitinib 2×5 mg/kg cohort, representing a significant attenuation of tyrosine hydroxylase destruction. This result was reproducible with western blotting, ELISA, and also immunohistochemistry assays (performed on 5 μM-thick coronal sections). These results therefore demonstrate a neuroprotective effect of masitinib in mice receiving masitinib treatment at 5 mg/kg twice per day and more generally proof-of-concept of a neuroprotective effect for inhibitors of mast cell function.

Several drugs are effective in the MPTP mouse model by virtue of their inhibitory activity on the enzyme monoamine oxidase B (MAO-B); for example, peroxisome agonist of proliferator-activated receptor or PPAR agonist. Indeed, the MAO-B enzyme transforms the MPTP into an active neurotoxic metabolite 1-methyl-4-phenylpyridinium (MPP+) which induces dopaminergic neuronal destruction. In order to further establish that the mechanism of protection of masitinib observed against MPTP induced toxicity was due to an anti-inflammatory action rather than MAO-B inhibition, the MAO enzymatic activity from total brain protein extract was quantified (Table 4).

TABLE 4
Monoamine oxidase activity detected by fluorometric method from total
brain protein extract after treatment by MPTP and masitinib.

| Treatment | Monoamine oxidase fluorometric assay |
|---|---|
| Control | 100 |
| MPTP | 108 |
| MPTP + Masitinib 2 × 5 mg/kg | 115 |

In all mice groups treated with MPTP alone or in combination with masitinib, the activity of monoamine oxidase was maintained with respect to the control group. Hence, one may conclude that the observed treatment-effect with 2×5 mg/kg masitinib is likely due to an anti-inflammatory action rather than MAO-B inhibition.

1.3. Conclusions

The results showed masitinib protection against the decrease expression of tyrosine hydroxylase. This was confirmed by using different methods of analysis such as western blot and ELISA test. These results indicate that masitinib might offer a protective role in the development of PSP. Moreover, no modification of monoamine oxidase enzymatic activity was observed, which appeared to show that the mechanism of protection of masitinib observed against MPTP induced toxicity was due to an anti-inflammatory action.

These preclinical findings provide proof-of-concept of masitinib's neuroprotective potential and more generally proof-of-concept of a neuroprotective effect for inhibitors of mast cell function. Masitinib is an effective targeted therapy against mast cells, exerting a direct proapoptotic, anti-migratory, and anti-activation action [Dubreuil et al., 2009, PLoS ONE; 4(9):e7258], thus, indirectly controlling the array of proinflammatory and vasoactive mediators these cells can release. Given that the neural pool of mast cells is influenced by their ability to rapidly cross the BBB, inhibition of mast cells peripheral to the BBB could therefore impact on the main pathological features of PSP. In conclusion, this study showed that masitinib, a potent and selective inhibitor mast cell activity, may be used as a potential treatment of PSP.

These results support initiation of human clinical trials.

### Example 2

### Clinical Study Protocol

Study design: Randomized, placebo-controlled, phase 2b/3 study to compare the efficacy and safety of masitinib versus placebo in the treatment of patients suffering from Progressive Supranuclear Palsy (PSP).

Diagnosis: Patients with probable or possible PSP.

Study treatment: Masitinib 100 and 200 mg tablets.

Associated product: Placebo, matching 100 mg and 200 mg tablets.

Duration of treatment: 48 weeks of study treatment with possible extension.

Main Inclusion Criteria:
Female or male patient aged between 40 and 80 years old, weighing more than 41 kg and with a Body Mass Index (BMI) between 18 and 35 kg/m².
Probable PSP (clinical signs of PSP) with PSP stage≦II defined as:
   at least a 12-month history of postural instability or falls during the first 3 years that symptoms are present; and
   PSPRS score <55; and
   an akinetic-rigid syndrome with prominent axial rigidity.
MAPT H1 positive haplotype.
Modified Hachinski score ≦3. This modified Hachinski will not include the focal neurological signs, symptoms or pseudobulbar affect questions, given the prominence of all 3 in PSP.
Score ≧25 on the mini-mental state examination (MMSE).
Patient judged by investigator to be able to comply with neuropsychological evaluation at baseline and throughout the study.
If the patient is receiving levodopa/carbidopa, levodopa/benserazide, a dopamine agonist, catechol-o-methyltransferase (COMT) inhibitor, or other Parkinson's medication, with the exception of Azilect (rasagiline), the dose must have been stable for at least 60 days prior to the screening visit and must remain stable for the duration of the study. No such medication can be initiated during the study. Subjects receiving rasagiline or CoQ10 must be on a stable dose for at least 90 days prior to the screening visit.

Main Exclusion Criteria:
Insufficient fluency in local language to complete neuropsychological and functional assessments.
Diagnosis of Amyotrophic Lateral Sclerosis or other motor neuron disease or other diagnosis of taupathies (such as corticobasal degeneration, frontotemporal lobe dementia, multiple system atrophy).
Presence of other significant neurological or psychiatric disorders including (but not limited to) Alzheimer's disease; dementia with Lewy bodies; prion disease; Parkinson's disease (which has not subsequently been revised to PSP); any psychotic disorder; severe bipolar disorder; seizure disorder; tumor or other space-occupying lesion; or history of stroke or head injury with loss of consciousness for at least 15 minutes within the past 20 years.
Structural abnormality on MRI that precludes diagnosis of PSP, such as cortical infarct in brain region that might account for subject's symptoms.
Patient with Feeding tube/recommendation for a feeding tube.
Patients with MPTP exposure.

Concomitant treatments: All medications taken by the patients at the onset of study and all medication given in addition to the Investigational Medicinal Product (masitinib) during the study are regarded as concomitant medications.
Patients must have been treated for a minimum of 2 months with a stable dose of levodopa and/or amantadine and/or rivastigmine at baseline, with no changes foreseen in therapy throughout the study.
Other drugs for the treatment of concurrent chronic diseases are allowed. However, their dosages should be kept constant throughout the study.
Vaccination is allowed except live attenuated vaccines.
If the patient is receiving levodopa/carbidopa, levodopa/benserazide, a dopamine agonist, catechol-o-methyltransferase (COMT) inhibitor, or other Parkinson's medication, with the exception of Azilect (rasagiline), the dose must have been stable for at least 60 days prior to the screening visit and must remain stable for the duration of the study. No such medication can be initiated during the study. Subjects receiving rasagiline or CoQ10 must be on a stable dose for at least 90 days prior to the screening visit.

Randomization:

Patients will be randomized in 2 groups (ratio 1:1). The randomization will be stratified according to:
Progression of PSPRS score (point/month) from date of first symptom to baseline, balanced between treatment groups in each of PSP patient populations “Normal progressors” (PSPRS score progression <1.3/month) and “Faster progressors” (PSPRS score progression >1.3/month);
PSPRS Score at Baseline;
Age at baseline.

Treatment administration: Patients enrolled will be randomised in 2 groups:
Group 1: patients will receive masitinib 3 mg/kg/day during 4 weeks then 4.5 mg/kg/day during 4 weeks and then 6 mg/kg/day (each switch being subjected to a toxicity control);
Group 2: patients will receive placebo with the same administration plan as masitinib.

Subjects randomized will receive a total daily dose of 3 mg/kg of masitinib or matching placebo during the first 4 weeks. The following rule will apply to define whether the dose of masitinib may be increase:
no severe suspected (or not assessable) adverse event was reported AND;
no suspected (or not assessable) adverse event led to treatment interruption AND;
no suspected (or not assessable adverse event) is ongoing at the time of the dose increase, regardless of its severity.

At the week 4 visit, if the patient does not present with a suspected or not assessable adverse event which was either severe, or leading to masitinib interruption, and if no suspected or not assessable adverse event is ongoing at week 4, regardless of its severity, the daily dose of masitinib will be increased to 4.5 mg/kg/day.

The patients presenting with non-severe suspected adverse event at the time of the dose increase can pursue the dose progression schedule with one-month delay. At the week 8 visit, if the patient does not present with a suspected or not assessable adverse event which was either severe, or leading to masitinib interruption, and if no suspected or not assessable adverse event is ongoing at week 8, regardless of its severity, the daily dose of masitinib will be increased to 6 mg/kg/day.

The dosage recommendations in case of adverse event mentioned in the management of toxicity section remain applicable anytime.

Dose of study treatment according to patient's weight are indicated in the tables below:

TABLE 5
Dose of study treatment (mg) according
to patient's weight (3 mg/kg/day).

3 mg/kg/day

| | Daily dose | Morning* | Evening** |
|---|---|---|---|
| Patient's weight in kg | (mg) | (mg) | (mg) |

| | | | | |
|---|---|---|---|---|
| >41.0 | ≦55.5 | 100 | | 100 |
| >55.5 | 77.7 | 200 | 100 | 100 |
| >77.7 | 99.9 | 300 | 100 | 200 |
| >99.9 | | 400 | 200 | 200 |

*Morning: the tablets should be taken during breakfast. In case of nausea, administration can take place during lunch.
**Evening: the tablets should be taken during dinner.

TABLE 6
Dose of study treatment (mg) according
to patient's weight (4.5 mg/kg/day).

4.5 mg/kg/day

| | Daily dose | Morning* | Evening** |
|---|---|---|---|
| Patient's weight in kg | (mg) | (mg) | (mg) |

| | | | | |
|---|---|---|---|---|
| >41.0 | ≦55.5 | 200 | 100 | 100 |
| >55.5 | 77.7 | 300 | 100 | 200 |
| >77.7 | 99.9 | 400 | 200 | 200 |
| >99.9 | | 500 | 200 | 200 + 100 |

TABLE 7
Dose of study treatment according
to patient's weight (6 mg/kg/day).

6 mg/kg/day

| | Daily dose | Morning* | Evening** |
|---|---|---|---|
| Patient's weight in kg | (mg) | (mg) | (mg) |

| | | | | |
|---|---|---|---|---|
| >41.0 | ≦55.5 | 300 | 100 | 200 |
| >55.5 | 77.7 | 400 | 200 | 200 |
| >77.7 | 99.9 | 500 | 200 | 200 + 100 |
| >99.9 | | 600 | 200 + 100 | 200 + 100 |

Criteria for Evaluation:

Primary Endpoint (Change from Baseline)

Absolute change from baseline of Progressive Supranuclear Palsy Rating Scale (PSPRS) score to Week 48. (This scale is in the public domain).

The PSPRS assesses 28 signs and symptoms of PSP in 6 categories: daily activities, behavior, bulbar, ocular motor, limb motor and gait/midline. Scores on the PSPRS range from a low of 0 (normal) to a high of 100 (most disability) [Golbe L I, et al. Brain. 2007; 130(Pt6):1552-6].

Secondary Variable (Change from Baseline)

Schwab and England Activities of Daily Living Scale (SEADL) scores to Week 48. (This scale is in the public domain).

The Schwab & England Activities of Daily Living Scale estimates the abilities of individuals living with a disease relative to a completely independent situation. The SEADL is an 11-point (0%, 10%, 20% . . . 100%; 100% is normal) ordinal scale, which measures overall disability based on a patient and informant interview. Scores range from 100%, which indicates a completely independent individual, and 0%, which indicates an individual who is no longer functioning [Schwab R S, England A C Jr. Projection techniques for evaluating surgery in Parkinson's Disease. pp.152-157; E. & S. Livingstone Ltd. (1969)]. A presentation of the SEADL would be similar to the following:
100% Able to do all chores without difficulty or impairment.
90% Able to do all chores but with some slowness (up to twice as long) or difficulty. Beginning to be aware of difficulty.
80% Able to do most chores but with some slowness (takes twice as long) and conscious of difficulty.
70% Difficulty and slowness doing chores, which take up to three or four times as long.
60% Can do most but not all chores, but with effort and very slowly; makes some mistakes.
50% Can do some chores but needs help with most; has difficulty with all and makes many mistakes.
40% Can help with a few chores but cannot do very many alone.
30% Can sometimes begin a few chores alone but cannot finish; needs much help.
20% Cannot do any chores alone (invalid).
10% Helpless; total dependence.
0% Vegetative; bedridden; loss of swallowing, bladder or bowel functions.
Cognitive function assessed via MMSE scores to Week 48 [Folstein M F, et al. (1975). Journal of psychiatric research 12 (3): 189-98]. (This scale is in the public domain).
Disease progression: assessed via:
   Brain volume changes, as measured by ventricular volumes, brain stem atrophy and frontal lobe volume measured by volumetric brain MRI to Week 48.
   Serum and cerebrospinal fluid Tau protein levels (CSF samples are optional and will be realized only for voluntary patients) to Week 48.
Severity of illness assessed via:
   Clinical Global Impression of Change (CGI-C) scores to Week 48 (This scale is in the public domain).
   Clinical Global Impression of Disease Severity (CGI-ds) scores to Week 48 (This scale is in the public domain).

Amongst the most widely used of extant brief assessment tools in psychiatry, the CGI is a 3-item observer-rated scale that measures illness severity (CGIS), global improvement or change (CGIC) and therapeutic response [Guy W, editor. ECDEU Assessment Manual for Psychopharmacology. 1976. Rockville, Md., U.S. Department of Health, Education, and Welfare].

The CGI is rated on a 7-point scale, with the severity of illness scale using a range of responses from 1 (normal) through to 7 (amongst the most severely ill patients). A presentation of the CGI-ds would be similar to the following:

Considering your total clinical experience with this particular population, how mentally ill is the patient at this time? 0=Not assessed; 1=Normal, not at all ill; 2=Borderline mentally ill; 3=Mildly ill; 4=Moderately ill; 5=Markedly ill; 6=Severely ill; 7=Among the most extremely ill patients.

The CGI-C scores range from 1 (very much improved) through to 7 (very much worse). A presentation of the CGI-C would be similar to the following:

Compared to his condition at admission to the project, how much has he changed? 0=Not assessed; 1=Very much improved; 2=Much improved; 3=Minimally improved; 4=No change; 5=Minimally worse; 6=Much worse; 7=Very much worse.
Psychiatric symptoms assessed via:
   Geriatric Depression Scale scores to Week 48 (This scale is in the public domain);
   Apathy Evaluation Scale scores to Week 48.

The Geriatric Depression Scale (GDS) has been tested and used extensively with the older population. The GDS Long Form is a brief, 30-item questionnaire in which participants are asked to respond by answering yes or no in reference to how they felt over the past week. A Short Form GDS consisting of 15 questions was developed in 1986. Questions from the Long Form GDS which had the highest correlation with depressive symptoms in validation studies were selected for the short version. Of the 15 items, 10 indicated the presence of depression when answered positively, while the rest (question numbers 1, 5, 7, 11, 13) indicated depression when answered negatively. Scores of 0-4 are considered normal, depending on age, education, and complaints; 5-8 indicate mild depression; 9-11 indicate moderate depression; and 12-15 indicate severe depression [Yesavage J A, et al. J Psychiatr Res 1983; 17:37-49].

The Apathy Evaluation Scale addresses characteristics of goal directed behavior that reflects apathy including behavioral, cognitive and emotional indicators. It consists of 18 items, with scores of 18-72 (higher scores reflect more apathy). Items are scored on 4-point Likert scale with descriptors for the “self” version (not at all true, slightly true, somewhat true, very true) and those for the clinician and informant version (not at all characteristic, slightly characteristic, somewhat characteristic, very characteristic). Two open questions are also asked (number of items reported, details offered in response to questions) to characterize apathy [Marin R S, et al. (1991). Psychiatry Research 38(2): 143-162].
Quality of life:
   PSP-QoL scale score to Week 48.

[Schrag A, et al. Measuring quality of life in PSP: the PSP-QoL. Neurology. 2006 Jul. 11; 67(1):39-44].

1. A method for the treatment of Progressive Supranuclear Palsy (PSP), wherein said method comprises administering to a patient in need thereof at least one mast cell inhibitor.

2. The method according to claim 1, wherein Progressive Supranuclear Palsy is the variant Richardson syndrome.

3. The method according to claim 1, wherein Progressive Supranuclear Palsy is the variant PSP-parkinsonism.

4. The method according to claim 1, wherein said patient has a progression of Progressive Supranuclear Palsy Rating Scale (PSPRS) of greater than or equal to 1.3 points per month.

5. The method according to claim 1, wherein said patient has a progression of Progressive Supranuclear Palsy Rating Scale (PSPRS) of less than 1.3 points per month.

6. The method according to claim 1, wherein Progressive Supranuclear Palsy is at a stage inferior or equal to a stage II.

7. The method according to claim 1, wherein said mast cell inhibitor is chosen from the group consisting of: masitinib, imatinib, cromolyn sodium, midostaurin, BLU-285, bosutinib, ibrutinib, LAS189386, DP-2618, fostamatinib, nilotinib, dasatinib, sunitinib, axitinib, pazopanib, and toceranib, or pharmaceutically acceptable salts or solvates thereof.

8. The method according to claim 1, wherein said mast cell inhibitor is masitinib or a pharmaceutically acceptable salt or solvate thereof.

9. The method according to claim 1, wherein said mast cell inhibitor is masitinib mesilate.

10. The method according to claim 1, wherein said mast cell inhibitor is administered at a daily dose of 1.0 to 12.0 mg/kg (mg per kg bodyweight).

11. The method according to claim 1 wherein said mast cell inhibitor is administered at an initial dose of 3.0 mg/kg/day during at least 4 weeks, then 4.5 mg/kg/day during at least 4 weeks, and at 6 mg/kg/day thereafter, with each dose escalation being subjected to toxicity controls.

12. The method according to claim 1, wherein said mast cell inhibitor is administered in two daily intakes.

13. The method according to claim 1, wherein said mast cell inhibitor is administered orally.

14. The method according to claim 1, wherein said mast cell inhibitor is administered in combination with at least one other pharmaceutically active ingredient.

15. The method according to claim 1, wherein said mast cell inhibitor is administered in combination with at least one other pharmaceutically active ingredient chosen from the group consisting of: levodopa, carbidopa-levodopa, dopamine agonists, monoamine oxidase B (MAO-B) inhibitors, catechol-O-methyl transferase (COMT) inhibitors, NMDA receptor antagonists, acetylcholinesterase inhibitors, and mixture thereof.

16. The method according to claim 1, wherein said mast cell inhibitor is administered in combination with at least one other pharmaceutically active ingredient chosen from the group consisting of: levodopa; carbidopa-levodopa; dopamine agonists chosen from bromocriptine, pergolide, pramipexole, ropinirole, cabergoline, apomorphine, and lisuride; MAO-B inhibitors chosen from safinamide, selegiline, and rasagiline; COMT inhibitors chosen from entacapone and tolcapone; NMDA receptor antagonists chosen from amantadine and memantine; acetylcholinesterase inhibitors chosen from rivastigmine, donepezil, and galantamine; and mixture thereof.

17. The method according to claim 1, wherein said mast cell inhibitor is administered in combination with at least one other pharmaceutically active ingredient in a combined preparation for simultaneous, separate, or sequential use.

18. A pharmaceutical composition comprising a mast cell inhibitor in combination with one or more pharmaceutically acceptable excipients, for use in a method for the treatment of Progressive Supranuclear Palsy.

19. A pharmaceutical composition according to claim 18, comprising a mast cell inhibitor and at least one other pharmaceutically active ingredient chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof, in combination with one or more pharmaceutically acceptable excipients.

20. A kit comprising a mast cell inhibitor, for use in a method for the treatment of Progressive Supranuclear Palsy.
